(19) United States
(12) Patent Application Publication
(10) Pub. No.: US 2023/0282333 A1
(43) Pub. Date: Sep. 7, 2023

(54) DEEP LEARNING-ASSISTED APPROACH FOR ACCURATE HISTOLOGIC GRADING AND EARLY DETECTION OF DYSPLASIA

(71) Applicant: UNIVERSITY OF MISSOURI AT COLUMBIA, Columbia, MO (US)

(72) Inventors: Deepthi S. RAO, Kansas City, MO (US); Praveen Ramesh RAO, Kansas City, MO (US)

(21) Appl. No.: 18/176,939

(22) Filed: Mar. 1, 2023

Related U.S. Application Data
(60) Provisional application No. 63/315,609, filed on Mar. 2, 2022.

(51) Int. Cl. G16H 30/20; G06N 3/09; G06N 3/047; G16H 50/20; G06T 7/00

(52) U.S. Cl. CPC G16H 30/20; G06N 3/09; G06N 3/047; G16H 50/20; G06T 7/0012; G06T 2207/20084; G06T 2207/20081; G06T 2207/30028; G06T 2207/30096; G06T 2200/24

(57) ABSTRACT

Methods and systems are provided for diagnosing early stages of inflammatory bowel disease (IBD), IBD severity predictions, and the preventions of invasive cancers associated with IBD. Deep learning methods are used for accurate histologic assessment of IBD in one or more target patients. In aspects, IBD associated dysplasia and adenocarcinoma is detected from histopathology data. For example, a deep learning model is modified and trained using a training data set comprising histopathology images to enable Bayesian deep learning. In aspects, the training data set may be labeled based on genetic and immunologic factors. A target patient can be diagnosed as having IBD at a particular severity level based on an output by the deep learned model. Additionally, a care plan for the target patient may be determined, organized, or modified based on the output provided by the deep learned model.

### CROSS-REFERENCE TO RELATED APPLICATIONS

This application claims priority to U.S. Provisional Application Ser. No. 63/315,609, filed on Mar. 2, 2022, the entire contents of which are incorporated by reference herein.

### BACKGROUND

Inflammatory bowel disease (IBD) encompasses chronic inflammatory state of the gastrointestinal (GI) tract and clinically presents in two forms: ulcerative colitis (UC) and Crohn's disease (CD). Unlike in traditional colon cancer, the process of cancer formation in IBD is more of a non-polypoid mucosal dysplasia, which leads to invasive cancer at an exaggerated rate. Assessment of disease activity is critical for developing and determining appropriate therapy in patients with IBD. For histologic measurements of disease activity to affect clinical decision making, pathologists should agree upon which standardized features to report and definitions of histologic healing and remission. However, the interobserver agreement is moderate at best.

### SUMMARY

At a high level, the technology relates to histological grading predictions for IBD and early detection of IBD associated dysplasia and adenocarcinoma using deep learning. In aspects, the present technology uses deep learning on images of IBD biopsy samples to provide accurate histological assessment associated with the histological grading predictions and the early IBD detection. For example, when a pathologist observes a biopsy sample under the microscope, the pathologist observes different kinds of patterns to make a diagnosis. In a similar fashion, the technology disclosed herein comprises software that enables accurate pattern recognition as well as improvements to the interobserver agreement. The accurate pattern recognition may be used for making diagnosis predictions associated with IBD.

In embodiments, the present technology will leverage Bayesian deep learning to model uncertainty in the predictions unlike conventional deep learning. In aspects, the disclosed technology may use ResNet, DenseNet, and EfficientNet architectures and insert additional layers of neurons (used during training) before the final fully-connected layer unlike conventional deep learning. Using Monte Carlo dropout during inference determinations, the present technology may form patch-wise classification in one or more histopathology images to identify the patterns.

In aspects, the present technology may utilize Bayesian deep learning and training using whole slide images of IBD biopsy samples. The training may include labeled data associated with the slide images.

This summary is intended to introduce a selection of concepts in a simplified form that is further described in the detailed description section of this disclosure. The summary is not intended to identify key or essential features of the claimed subject matter, nor is it intended as an aid in determining the scope of the claimed subject matter. Additional objects, advantages, and novel features of the technology will be set forth in part in the description that follows, and in part will become apparent to those skilled in the art upon examination of the disclosure or learned through practice of the technology.

### BRIEF DESCRIPTION OF THE DRAWINGS

The present technology is described in detail below with reference to the attached drawing figures, wherein:

FIG. 1 is a block diagram of an example operating environment suitable for implementations of the present disclosure;

FIG. 2 is a diagram depicting an example computing architecture suitable for implementing aspects of the present disclosure;

FIG. 3 is a flow diagram of an example method for predicting disease activity using deep learning, in accordance with aspects described herein;

FIG. 4 is a flow diagram of an example method for predicting disease activity using deep learning, in accordance with aspects described herein;

FIG. 5 is an example flowchart for predicting disease activity using deep learning, in accordance with aspects described herein; and

FIG. 6 is an example computing device suitable for implementing the method illustrated in FIG. 1, in accordance with an embodiment described herein.

### DETAILED DESCRIPTION

Histological grading predictions for inflammatory bowel disease (IBD) and early detection of IBD associated dysplasia and adenocarcinoma, using deep learning, are disclosed. Accurate histologic grading of IBD results in improvements to the interobserver agreement. Accurate histologic grading of IBD results and early detection of IBD associated dysplasia and adenocarcinoma, using deep learning, can result in reduced histologic remission and lower rates of clinical relapse. In turn, patients having accurate histologic grading of IBD results and early detection of IBD achieve substantial improvements in the quality of life. Further, embodiments of the present technology can also lead to optimization of maintenance therapy, decreased rates of corticosteroid use, and reduced exacerbated hospitalization in IBD patients. Further, aspects of the present technology result in more precise clinical management of patients at the individual patient level.

Aspects of the presently disclosed technology include a deep learning-assisted approach for accurate histologic assessments of IBD and early detection of IBD associated dysplasia and adenocarcinoma. For example, embodiments may include (a) the generation of labeled data for deep learning, (b) Bayesian deep learning (BDL) for accurate histologic assessment of IBD severity, and (c) BDL for early detection of IBD associated dysplasia and adenocarcinoma. The technology disclosed herein enables accurate histologic grading of IBD resulting in higher rates of histologic remission with lower rates of clinical relapse, thus resulting in substantial improvement in patient care quality. Aspects of the present technology also enable early detection of IBD associated dysplasia and lead to decreased rates of adenocarcinoma.

IBD encompasses chronic inflammatory state of the gastrointestinal (GI) tract and clinically presents in two forms: ulcerative colitis (UC) and Crohn's disease (CD). The annual prevalence rate of IBD in the United States is 201/100,000 adults. Healthcare facilities have sizable populations of patients with IBD who have been seen in the GI clinic of the healthcare facility. Data from an internal review of the GI clinic shows that from March 2016 to July 2020, the GI clinic has seen 474 IBD patients, which includes 277 (58.4%) females and 194 (42.6%) males. There were 253 (53.4%) patients with a diagnosis of Crohn's disease and 197 (41.5%) with ulcerative colitis remainder of the cases are still under investigation or are deemed indeterminate colitis.

Etiology is not always clearly associated with the disease, however, several factors may be determined to be associated with its etio-pathogenesis including genetic and immunologic factors, environmental influences, or alteration in luminal gut microbiome. Some complications of IBD include development of bowel perforation, bowel obstruction, fistulization of bowels and GI bleeding, to name a few. One of the major complications of IBD involving the colon and/or rectum is the risk of development of colorectal cancer. Unlike in traditional colon cancer, the process of cancer formation in IBD is more of a non-polypoid mucosal dysplasia, which leads to invasive cancer at an exaggerated rate.

It is desirable to accurately assess disease activity for developing and determining appropriate therapy in patients with IBD. In embodiments, disease activity and treatment response may be assessed using symptoms, biomarkers, endoscopy, and histology. In some embodiments, the disease activity and treatment response may be assessed using clinical and endoscopic measures. In some aspects, mucosal biopsy specimens are obtained to diagnose IBD and to assess inflammation. In some aspects, histology is used as an independent prognostic factor for identifying disease activity and a corresponding treatment response.

Patients in clinical remission often have endoscopic and histologic activity. As such, other IBD management systems may focus on mucosal healing referring primarily to endoscopic remission instead of symptom improvement. However, histologic activity may be present in up to 40% of patients with a normal-appearing mucosa on endoscopy, and recent evidence including meta-analysis of 15 studies with 1573 patients indicates that achievement of histologic remission, compared with endoscopic or clinical remission may be superior and is strongly associated with lower rates of clinical relapse, corticosteroid use, hospitalization, and neoplasia. Therefore, in some embodiments of the presently disclosed technology, histologic remission is a treatment goal for patients having IBD. Further, in some embodiments, deep learning using patient data from IBD patients having an association between residual histologic activity and clinical relapse is utilized for determining accurate histologic assessments of IBD and early detection of IBD associated dysplasia and adenocarcinoma.

Patient care plans for IBD patients, in some embodiments, are based on histologic measurements of disease activity associated with the patient. Continuing the example, a plurality of pathologists agreed upon which standardized features to report and definitions of histologic healing and remission. As such, the histologic measurements of disease activity associated with the training data is standardized in this way.

Although 30 histologic scoring systems in IBD have been described, only 3 have undergone extensive validation: the Geboes' score (GS), Nancy Index (NI), and Robarts Histopathologic Index (RHI). However, even with one of these extensively validated indices, the interobserver agreement is moderate at best. To solve this problem with the interobserver agreement, the disclosed deep learning-assisted approach for accurate histologic assessment of IBD and early detection of IBD associated dysplasia and adenocarcinoma includes employing Bayesian deep learning along with state-of-the-art deep networks for image classification. This approach enables achievement of high accuracy determinations as well as determinations of uncertainty in the model predictions.

Deep learning may be used for early detection of cancers to predictions of disease survivability. For example, deep learning may be used for early detection of IBD associated with dysplasia and adenocarcinoma. As a result, the early detection may be used for improved patient management, improved clinical outcomes, and reduced healthcare costs.

In aspects, an artificial neural network (ANN) may be used for the predictions associated with IBD detection. For example, the ANN may include an input layer, hidden layers, and an output layer. The hidden layer may be modeled as neurons that represent one or more mathematical functions. Hardware, such as a graphical processing unit, may also be used. A large number of the hidden layers may also be used. Pattern recognition in histopathology images using a deep neural network may comprise multiple representations of the input images at different levels of abstraction. In embodiments, the system can learn complex, non-linear decision boundaries for achieving high accuracy in classification tasks. In addition, the system can avoid the tedious process of hand-engineered feature selection required by conventional machine learning techniques. Some of the deep networks for the image classification can include use of ResNet, Inception-v4, InceptionResNet, ResNeXt, DenseNet, EfficientNet, and FixEfficientNet.

Various aspects of neoplastic and non-neoplastic gastrointestinal, pancreatobiliary and hepatic pathology conditions associated with IBD may be analyzed. In aspects, a deep learning approach for building the deep learning model may comprise using only a few training samples of each class associated with IBD during training. This approach has achieved 95% accuracy on the tested dataset provided by one or more expert pathologists. Among the total number of IBD patients used for training, some of the training data may include tissue samples during colonoscopies.

In an aspect, labeled data may be generated for deep learning training, validation, and testing using histopathology images of IBD patients. IBD patients may require frequent colonoscopies to evaluate the disease, and these endoscopic procedures include biopsies of the intestines. The patients are assessed for symptoms of IBD and for appropriate management of their disease. Colonoscopies are performed and biopsies are taken for further detailed microscopic evaluation, and the histopathologic diagnosis along with the activity status, grading of the severity, and the evaluation for the presence of IBD associated dysplasia and the assessment of response to ongoing therapy are rendered. The whole slide images (WSIs) of tissue samples may be obtained using a scanner. The WSIs are then labeled at the microscopic level to define the characteristic features such as architectural distortion, neutrophilic infiltrate, cryptitis, crypt abscess, basal lymphoplasmacytosis, presence or absence of characteristics such as paneth cell metaplasia, granuloma, and dysplasia and further grade the level of inflammatory response according to a standard histologic scoring system. In one embodiment, a labeled dataset with nearly 100,000 image patches (512×512 pixels) may be constructed.

Further, identifiable biospecimens (pathology slides), that were previously collected for the purpose of patient care may be used. These slides may be used for comparison between an AI approach and for decision making using WSIs compared to that of a pathologist using traditional microscopy and glass slides. An IRB waiver under the exempt category may be used based on the identifiable information of the biospecimens (specifically, the pathology case number, patient name, and other identifying information on the pathology slide) being de-identified. To protect the PHI (identifiers) from improper use and disclosure, the identified data may only be used during the initial case selection phase, after which de-identified data will be used for the remaining data collection, deep learning and analysis portions of the method. Additionally, for methods comparison, the identifiable information may be classified under secondary research for which consent is not required. Some patients are not contacted initially or in any subsequent longitudinal or long-term follow up.

In further embodiments, a system may be provided herein having a processor and one or more computer storage media, which causes the processor to perform a method. The method comprising accessing a training data set of histopathology images from one or more patients having TBD. Additionally, they methods provides for training a deep learning model with the training data set to generate a trained model, the trained model configured to classify a histopathology image and processing, via the trained model, the histopathology image of a target patient. Further, the method may provide, based on processing the histopathology image of the target patient, a histologic assessment determination associated with an IBD severity for display via a user interface.

In yet another embodiment, a method may be provided herein. The method comprising accessing a training data set of histopathology images from one or more patients having IBD. Additionally, they methods provides for training a deep learning model with the training data set to generate a trained model, the trained model configured to classify a histopathology image and processing, via the trained model, the histopathology image of a target patient. Further, the method may provide, based on processing the histopathology image of the target patient, a histologic assessment determination associated with an IBD severity for display via a user interface.

In another embodiment, one or more non-transitory computer storage media having computer-executable instructions embodied thereon, that when executed by at least one processor, cause operations comprising accessing a training data set of histopathology images from one or more patients having IBD. The media further causes the training a deep learning model with the training data set to generate a trained model, the trained model configured to classify a histopathology image and processing, via the trained model, of the histopathology image of a target patient. Further, the media may provide, based on processing the histopathology image of the target patient, a histologic assessment determination associated with an IBD severity for display via a user interface.

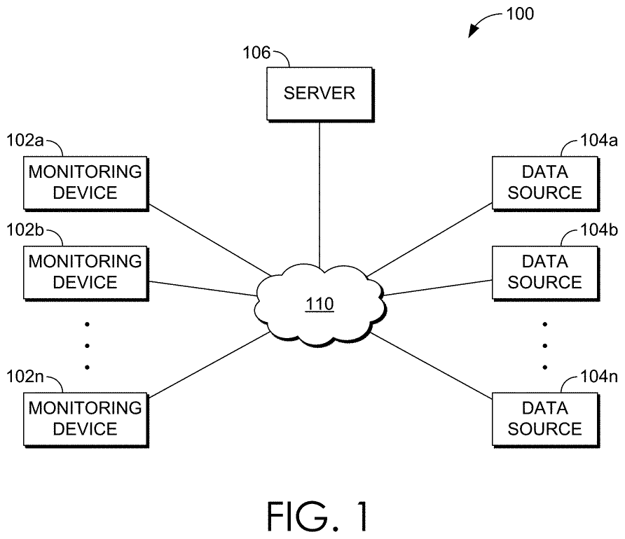

Turning now to FIG. 1, a block diagram is provided showing an example operating environment in which some embodiments of the present disclosure may be employed. It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether for the sake of clarity. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, some functions may be carried out by a processor executing instructions stored in memory.

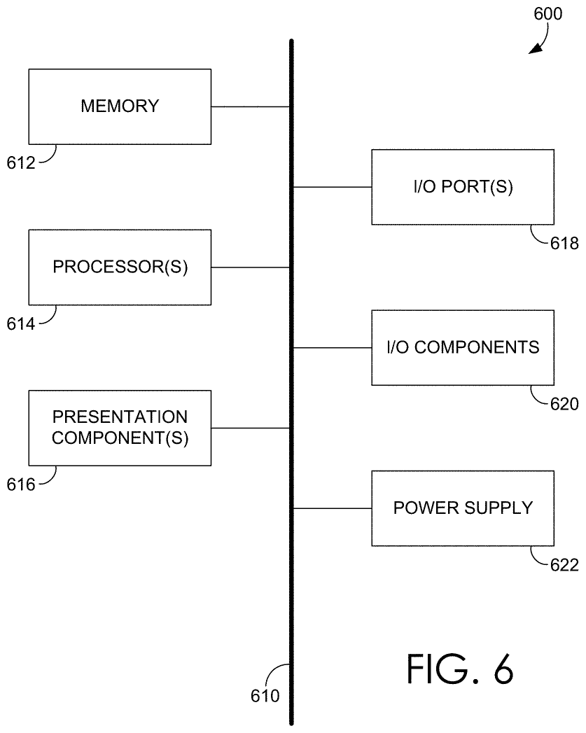

Among other components not shown, example operating environment 100 includes a number of monitoring devices, such as monitoring devices 102a and 102b through 102n; a number of data sources, such as data sources 104a and 104b through 104n; server 106; and network 110. It should be understood that environment 100 shown in FIG. 1 is an example of one suitable operating environment. Each of the components shown in FIG. 1 may be implemented via any type of computing device, such as computing device 600 described in connection to FIG. 6, for example. These components may communicate with each other via network 110, which may include, without limitation, one or more local area networks (LANs) and/or wide area networks (WANs). In some implementations, network 110 comprises the Internet and/or a cellular network, amongst any of a variety of possible public and/or private networks.

It should be understood that any number of user devices, servers, and data sources may be employed within operating environment 100 within the scope of the present disclosure. Each may comprise a single device or multiple devices cooperating in a distributed environment. For instance, server 106 may be provided via multiple devices arranged in a distributed environment that collectively provide the functionality described herein. Additionally, other components not shown may also be included within the distributed environment.

Monitoring devices 102a and 102b through 102n may be client devices on the client-side of operating environment 100, while server 106 may be on the server-side of operating environment 100. Server 106 can comprise server-side software designed to work in conjunction with client-side software on monitoring devices 102a and 102b through 102n so as to implement any combination of the features and functionalities discussed in the present disclosure. This division of operating environment 100 is provided to illustrate one example of a suitable environment, and there is no requirement for each implementation that any combination of server 106 and monitoring devices 102a and 102b through 102n remain as separate entities.

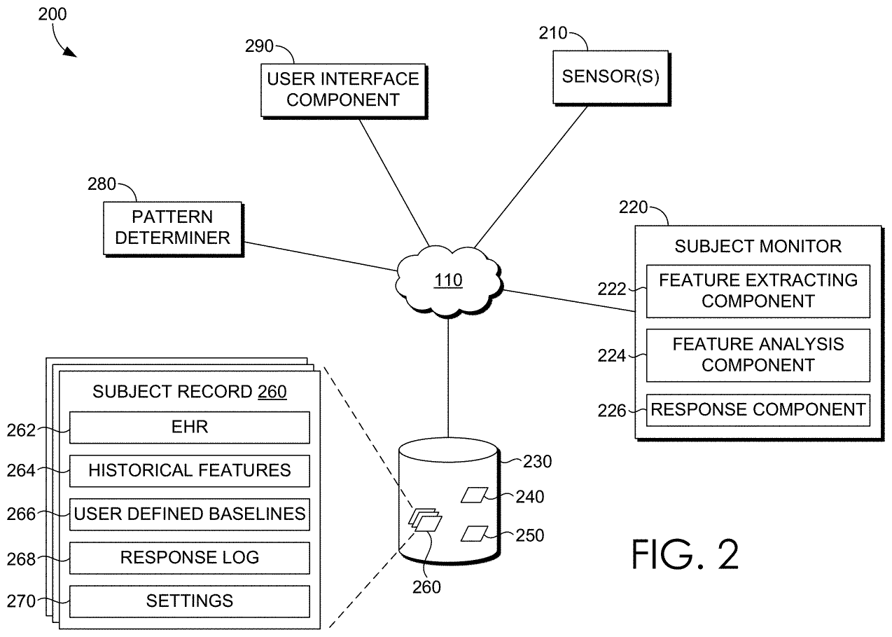

Monitoring devices 102a and 102b through 102n may comprise any type of electronic device with one or more sensors operable to acquire clinical or physiological information about a subject, such as various types of physiological measurements, physiological variables, or similar clinical information associated with a particular physical or mental state of the subject. The one or more sensor components, as discussed in detail with respect to sensor 210 in FIG. 2, may comprise a subject-wearable sensor component or a sensor component integrated into the subject's environment. Examples of sensor components of the monitoring devices include a sensor positioned on an appendage (on or near the subject's head, attached to the subject's clothing, worn around the subject's head, neck, leg, arm, wrist, ankle, finger, etc.); a skin-patch sensor adhered to the subject's skin, ingestible or sub-dermal sensor; a sensor components integrated into the subject's living environment (including the bed, pillow, or in the subject's bathroom); and sensors operable with or through a smartphone carried by the subject, for example. It is also contemplated that the clinical or physiological information about the subject, such as monitored variable and/or clinical narratives, may be received from human measurements, human observations, or automatically determined by sensors in proximity to the patient. In one embodiment, the monitoring device comprises a plethysmophgrahic wristband sensor or wearable ECG, which may be carried out using a fitness tracker wristband device or mobile device.

Data sources 104a and 104b through 104n may comprise data sources and/or data systems, which are configured to make data available to any of the various constituents of operating environment 100, or system 200 described in connection to FIG. 2. (For example, in one embodiment, one or more data sources 104a through 104n provide (or make available for accessing) subject data to the subject monitor 220 of FIG. 2.) Data sources 104a and 104b through 104n may be discrete from monitoring devices 102a and 102b through 102n and server 106, or may be incorporated and/or integrated into at least one of those components. In one embodiment, one or more of data sources 104a though 104n comprise one or more sensors be integrated into or associated with one or more of the monitoring device(s) 102a, 102b, or 102n or server 106.

Operating environment 100 can be utilized to implement one or more of the components of system 200, described in FIG. 2, including components for collecting subject data, extracting features, determining patterns, analyzing features, and initiating responses to provide an improved method for early detection of clinical conditions. In many instances herein, the term features describe aspects of images of a biopsy taken by one or more sensors. For example, biopsy images are taken of colon samples for a patient. Those samples are analyzed for particular features that may be normal or abnormal and indicative of a complication such as IBD.

Referring now to FIG. 2, and referring to FIG. 1, a block diagram is provided showing aspects of an example computing system architecture suitable for implementing an embodiment and designated generally as system 200. System 200 represents only one example of a suitable computing system architecture. Other arrangements and elements can be used in addition to or instead of those shown, and some elements may be omitted altogether for the sake of clarity. Further, as with operating environment 100, many of the elements described herein are functional entities that may be implemented as discrete or distributed components or in conjunction with other components, and in any suitable combination and location.

Example system 200 includes network 110, which is described in connection to FIG. 1, and which communicatively couples components of system 200 including sensor(s) 210, subject monitor 220, pattern determiner 280, a user interface component 290, and storage 230. Subject monitor 220 (including its components 222, 224, and 226) and pattern determiner 280 may be embodied as a set of compiled computer instructions or functions, program modules, computer software services, or an arrangement of processes carried out on one or more computer systems, such as computing device 600 described in connection to FIG. 6, for example.

In one embodiment, the functions performed by components of system 200 are associated with one or more of sensors 210. In particular, the sensors 210 may be integrated with or otherwise associated with one or more user devices (such as monitoring device 102a). Further, the components of system 200 may be distributed across one or more monitoring devices and servers, or be implemented in the cloud. Moreover, in some embodiments, these components of system 200 may be distributed across a network, including one or more servers (such as server 106) and client devices, in the cloud, or may reside on a user device (such as monitoring device 102a). Moreover, these components, functions performed by these components, or services carried out by these components may be implemented at appropriate abstraction layer(s) such as the operating system layer, application layer, hardware layer, etc., of the computing system(s). Alternatively, or in addition, the functionality of these components and/or the embodiments described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. Additionally, although functionality is described herein with regards to specific components shown in example system 200, it is contemplated that in some embodiments functionality of these components can be shared or distributed across other components.

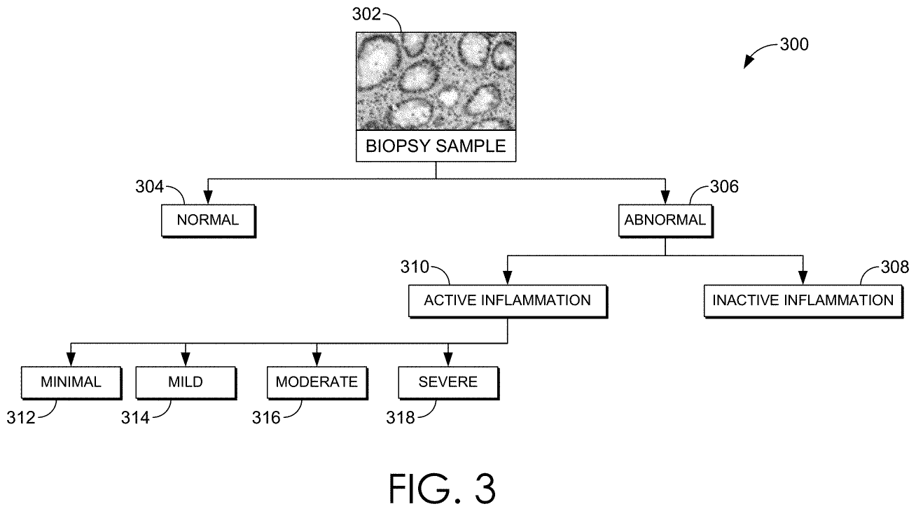

Continuing with FIG. 2, one or more sensors 210 are generally operable to acquire clinical or physiological information about a subject. Physiological information includes information relating to the functioning of the subject's body. Various types of physiological measurements, physiological variables, or similar clinical information associated with a particular physical state of the subject may be acquired. The sensors 210 may be used to analyze samples acquired and delivered to the sensors 210. For example, the sensors 210 may be one or more cameras or image sensors associated with a microscopy system. The sensors 210 may also be associated with one or more systems that are able capture images of biopsy samples such as biopsy sample 302 in FIG. 3.

The one or more sensors 210 may include one or more image capture sensors and processors operable for providing electrical signals corresponding to the images of one or more biopsy samples of various conditions or states of a subject. The electrical signals captured by the sensors 210 correspond the images that may use traditional microscopy or any other form of imaging of biopsy samples.

The histopathology data, or biopsy data, acquired by the sensors 210 is used by the subject monitor 220. The subject monitor 220 is generally responsible for monitoring the subject's biopsy data for information that may be used in determining information about the subject's clinical condition, which may include identifying and/or tracking features. In an embodiment, the subject monitor 220 collects the raw biopsy information, or histopathology images, from a sensor 210 and performs signal processing, such as patch-wise classification in the histopathology images to identify patterns.

The subject monitor 220 may operate through one or more components, including a features extracting component 222, a features analysis determiner, 224, and a response component 226. The features extracting component 222 may be responsible for identifying one or more features from the histopathological data acquired from the sensors 210. Features may be inherent in or interpreted from raw data. Raw data, or primary data, is data collected directly from the sensors without manipulation.

In some embodiments, the data relating to features identified by the features extracting component 222 is provided to a pattern determiner 280. The pattern determiner 280 may use features data to establish a baseline for one or more features. This baseline may be stored as a subject-defined baseline or a baseline for general histopathological data or biopsy samples. The baseline may be later used to analyze current features data in determining the subject's clinical condition such as abnormalities in biopsy samples or histopathological data. In some embodiments, after establishing a baseline, the pattern determiner 280 continues to receive the features data and can update the baseline if the newly received data, in combination with the previously received data, supports a different baseline value. In doing so, the pattern determining 280 may be able to distinguish between a temporary change in a features image, which would not necessarily require updating a subject-defined baseline, and a more permanent change in the feature image.

After the features extracting component 222 identifies features or abnormalities, the features analysis component 224 analyzes the features to provide useful information about the subject's clinical condition. Upon performing an analysis, the features analysis component 224 may determine a probability that the subject has a clinical condition. A clinical condition or state includes a disease, diagnosis, or other medical event indicating illness or injury, as described in further detail below. The probability of a clinical condition may be a probability that the subject presently has a condition. The probability may also relate to a future condition, such as an acute risk that the subject will develop sepsis in the immediate future.

The features analysis component 224 may analyze the features found within biopsy samples or histopathological data by using a feature baseline. A baseline is an initial known sample of a biopsy or histopathological dataset used for comparison of current feature values. The baseline may be an image or a pattern. Further, the baseline may be for a “normal” or healthy state of a feature or may be for an “abnormal” or unhealthy state for that feature. There may also be a multiple normal or abnormal baselines for a particular feature.

As previously mentioned, the baseline may be a subject-defined baseline determined by a pattern determiner 280 or it may be generically defined. Using a subject-defined baseline may increase the sensitivity of the system 200, allowing clinical conditions to be predicted more quickly and accurately. In other embodiments, the baseline used is a population-based baseline or one defined by a population other than the subject.

In embodiments, the features analysis component 224 uses the baseline, either a subject-defined baseline or a population-based baseline, in its analysis by performing a statistical comparison of the current images for a feature and the baselines images. The comparison may be either between the current feature image and one baseline image, such as a normal baseline or abnormal baseline, or may be between the current image and both a normal baseline and an abnormal baseline. In some embodiments, the baseline is compared to a mean image of multiple current feature images taken over a period of time. Additionally, the comparison may first comprise determining that the current image, or current mean image, for the image deviates from the baseline. And if a deviation is detected, then determining if the deviation is statistically significant such that the subject may have a clinical condition. Accordingly, a probability that the subject has a clinical condition may represent a statistical similarity between a current feature image and one or more baselines.

Upon determining the subject's clinical condition, the response component 226 may determine and initiate a response. The response may be one that is appropriate based on the determined probability of a clinical condition. A response determined and initiated by the response component 226 may include providing an alert, notification, or warning that the subject may have a clinical condition, providing recommended directions, automatically rescheduling resources such as reserving a hospital bed, scheduling transportation for the subject to a hospital or a quarantine area, or schedule a consult with a provider. The response may be performed with respect to the subject, the subject's healthcare provider, or another designated third party. For example, an alert or warning may be sent to the subject or, if the subject is a solider on active duty, to the subject's commanding officer as a designated third party.

The subject component 220 as well as other components of the system 200 may be communicatively coupled to storage 230. Storage 230 generally stores information including data, computer instructions (e.g., software program instructions, routines, or services), logic, profiles, and/or models used in embodiments. In an embodiment, storage 230 comprises a data store (or computer data memory). Further, although depicted as a single data store component, storage 230 may be embodied as one or more data stores or may be part of a cloud-based platform.

As shown in the example system 200, storage 230 includes population-based baselines 240, features logic 250, and one or more subject records 260. Population-based baselines 240 may include one or more baselines used for determining a probability that the subject has a clinical condition that is based on a population greater than the subject. Many different populations may be used to define a population-based baseline 240. A population-based baseline may be defined by information relating to the general population or may be tailored to a population having similarities with the subject. For example, a population-based baseline 240 may be defined by a population having similar demographics as the subject or having a similar medical history. Similar demographics may include age, sex, and weight. Additionally, the population-based baseline 240 may be based on a geographic location. For example, a population-based baseline for an American soldier stationed in Iraq may be based on a population of people living in the Middle East. The baseline could be even more specific to the subject and be based on a population of other American soldiers stationed in Iraq. Similar demographics may be combined with other population-based filters, such as geographic location. A population-based baseline may be provided by external sources or may be generated by the pattern determiner 280 based on data obtained from sensors associated with other subjects.

Storage 230 also includes features logic 250. Features logic 250 may include rules, conditions, associations, classification models, algorithms, or other criteria to identify features, determine patterns, and/or identify probabilities of a clinical condition. For example, in one embodiment, features logic 250 may include comparing the current features values with a feature baseline to determine that the current feature values are abnormal. The features logic 250 can take many different forms. For example, the features logic 250 may use machine-learning mechanisms to determine feature similarity, or other statistical measures to determine the physiological data acquired belongs to a particular feature or set of features.

In some embodiments, the features logic 250 provides a manner of determining an associated confidence score regarding the strength of the determined probability that the subject has a clinical condition. Additionally, an associated confidence score may be determined for a feature pattern or baseline determined by the pattern determiner 280. The confidence score may be based on the strength of the pattern, which may be determined based on the number of observations used to determine the pattern, the age or freshness of the observations, and the similarity between the baseline and what is expected based on a population-defined feature baseline. The confidence score may be used when calculating the probability the subject has a clinical condition and/or determining appropriate responses to initiate.

Features logic 250 may be updated on a periodic or as-needed basis to provide up-to-date information relating to possible clinical conditions and features. For example, as additional clinical conditions are identified or as additional indicators of known clinical conditions are determined, the features logic 250 may be updated to include new rules, associations, algorithms, or other criteria to allow the present disclosure to detect the clinical conditions based on the additional indicators. Updates to the features logic 250 may also include updates to existing information relating to clinical conditions being detected.

Lastly, storage 230 may also include one or more subject records 260. An example subject record 260 is provided in FIG. 2. The example subject record 260 includes an electronic health record (EHR) 262, historical features 264, subject-defined baselines 266, response log 268, and settings 270. The information stored in subject record 260 may be available to the subject monitor 220 or other components of the system 200.

An EHR 262 associated with the subject may include the subject's demographic information, past medical history, family medical history, medications the subject is taking, and immunizations. Additionally, the EHR 262 may include information relating to the subject's clinical condition and care for the condition, including a diagnosis, a treatment plan, clinical notes, orders, images, and other electronic documents relating to the subject's health. This information may be used to set baselines for the subject, including subject-defined and population-defined baselines, and may be used by the response component 226 to determine a response.

Much of the information contained in storage 230 or determined by another component of the system 200 may be accessible to a subject through the user interface component 290. The user interface component 290 may display information relating to the system 200, provide access to information, and receive inputs. The user interface component 290 may be designed to be used by the subject who is being monitored. The user interface component 290 may also be configured for use by a healthcare provider or other third party having rights or privileges to access the subject's physiological data.

An embodiment of the user interface component 290 takes the form of a user interface and application, which may be embodied as a software application operating on one or more mobile computing devices, tablets, smartphones, front-end terminals in communication with back-end computing systems, laptops, or other computing devices. In an embodiment, the user interface component 290 includes a Web-based application or set of applications usable to manage subject services provided by an embodiment of the invention. For example, in an embodiment, user interface component 290 facilitates processing, interpreting, accessing, storing, retrieving, and communicating information acquired from the sensor 210, patient monitor 220, pattern determiner 280, and storage, such as the subject record 240, including probable clinical conditions determined by embodiments of the invention as described. In some embodiments, the user interface component 290 may be a part of a wearable monitoring device with a sensor 210.

A subject application facilitates accessing and receiving information from the sensors 210 and/or the patient monitor 220. Embodiments of the user interface component 290 also facilitates accessing and receiving information from a subject about including medical history; family history, demographics, etc. The user interface component 290 may also facilitate accessing and receiving information from a healthcare provider or third party such as healthcare resource data; variables measurements, results, recommendations, or orders, for example. The user interface component 290 may also be used for providing diagnostic services or evaluation of the performance of various embodiments.

As previously mentioned, embodiments of the user interface component 290 display one or more responses initiated by the response component 226. In an embodiment, the user interface component 290 receives a notification (such as an alarm or other indication) from the response component 226 through network 110 and displays the notification to the subject, a healthcare provider or a third party. The user interface component 290 may be used to facilitate access by a subject to functions or information on the subject monitor 220, such as operational settings or parameters, subject identification, subject data stored, and diagnostic services or firmware updates for the subject monitor 220, for example.

Further, embodiments of the user interface component 290 may be operable to receive one or more inputs from the subject or other party accessing the user interface component 290. For example, the subject may use the user interface component 290 to provide an acknowledgement of a response, such as a warning that the subject has a probability of a clinical condition. The acknowledgment by the subject may cause the user interface component 290 to send a signal to the response component 226 indicating the acknowledgment, which may prevent further responses to be initiated from the response component 226. Such acknowledgments may be used to prevent redundant responses. The user interface component 290 may also receive input from a subject or other person to set preferences or adjust settings.

Though not illustrated, the example environment 200 may further include one or more security components, such as a firewall. One or more of the components in the environment 200 may be communicatively coupled to the network 110 from behind a security component, such as a firewall. For example, a firewall may be associated with subject monitor 220, pattern determiner 280, subject record 260, EHR 262, historical features 264, user-defined baselines 266, and other components that may contain personal identification or health information about the subject. Such a firewall may reside between a component of the environment 200 and the network 110, such as on a server (such as server 106 in FIG. 1), or may reside on or as part of a component. Additionally cyber security features may be a part of the security component.

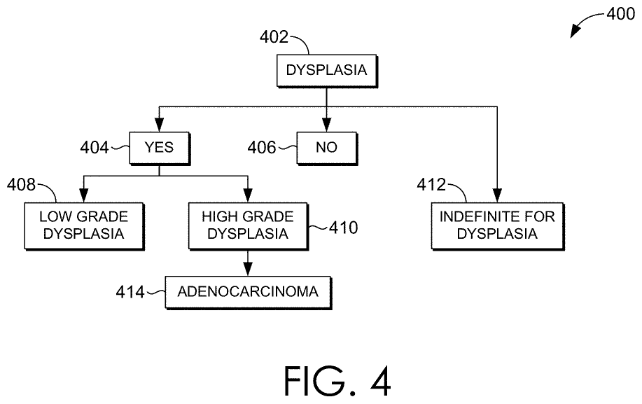

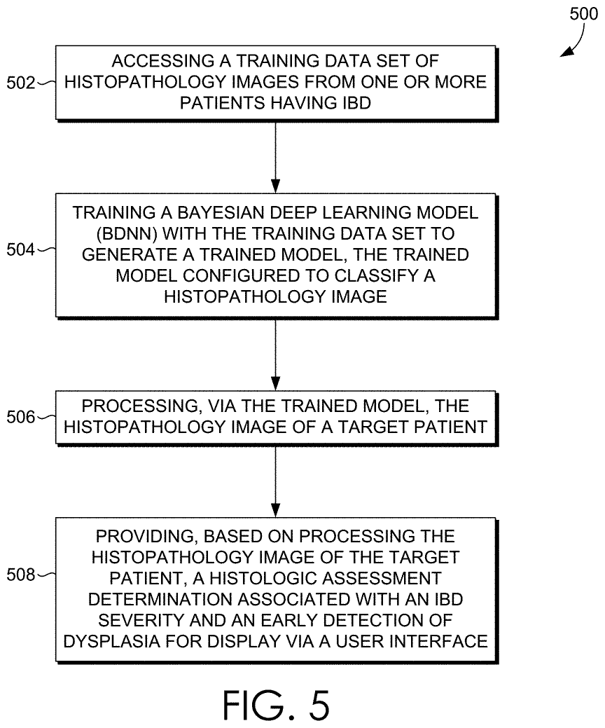

FIG. 3 provides an example method that may be performed using the computing device of FIG. 6, and is suitable for achieving the described advantages based on detecting disease activity associated with IBD in one or more patients. In embodiments, one or more computer storage media having computer-executable instructions embodied thereon that, when executed, by one or more processors, cause the one or more processors to perform operations illustrated in FIG. 3. Each block or step of method 300 and other methods described herein comprises a computing process that may be performed using any combination of hardware, firmware, and/or software. For instance, various functions may be carried out by a processor executing instructions stored in memory. The methods may also be embodied as computer-usable instructions stored on computer storage media. The methods may be provided by a stand-alone application, a service or hosted service (stand-alone or in combination with another hosted service), or a plug-in to another product, to name a few. Accordingly, method 300 may be performed by one or more computing devices, such as a smartphone or other user device, a server, or by a distributed computing platform, such as in the cloud. Additionally, the methods described in FIG. 3, FIG. 4, and FIG. 5 may be performed and use components described with respect to FIG. 1 and FIG. 2.

As illustrated in FIG. 3, example method 300 provides steps for predicting disease activity associated with IBD using deep learning. Example method 300 provides for enabling deep learning-assisted diagnostics. FIG. 3 shows the decision-making process of a system for IBD cases. Starting with a biopsy sample 302, the system observes different kinds of patterns to make a diagnosis. At each stage, pattern recognition is done by the system via deep learning to enable accurate pattern recognition and to improve the interobserver agreement. The system leverages Bayesian deep learning, which will enable the modeling of any uncertainty in the predictions. A ResNet/DenseNet/EfficientNet architecture is modified by inserting a Dense layer followed by Dropout layer (used during training) before the final fully-connected layer to enable Bayesian deep learning, in some embodiments. The modified model referred to hereinafter as Bayesian deep neural network (BDNN) is then trained on input data.

ResNet employs residual connections between hidden layers for accurate image classification. DenseNet has multiple connections to next layers/units in the network. EfficientNet is designed to achieve both high efficient and accuracy using a scaling method. Using Monte Carlo dropout during inference on the trained model, the method performs patch-wise classification in the histopathology images to identify the desired patterns. From the labeled dataset, 70% is used for model training in some embodiments. Continuing the example, 15% of the labeled dataset is used for model validation, and 15% of the labeled dataset is used for model testing. Data augmentation techniques such as resize, random left/right flip, rotation, and others may also be used. In embodiments, 90% or higher accuracy may be achieved for the test set.

Biopsy data 302 is received from the one or more sensors. The biopsy data 302 may be produced as a microscopy image. The image is processed and analyzed using the above trained method and may determine that the biopsy data 302 is normal 304. The trained model of method 300, may compare the biopsy data 302 with labeled data or baseline data to determine that the biopsy data 302 is normal 304. In another example, the trained model follows a decision tree such as method 300 to determine the nature of the biopsy data 302. If the trained model determines that the biopsy data 302 is abnormal 306 compared to the baseline set of biopsy images, the method will continue to determine if the biopsy data 302 shows signs of inflammation. The trained model of method 300 will compare the biopsy data 302 with baseline data and determine if the sample shows active inflammation 310 or inactive inflammation 312.

Upon determination of the inflammation status, the trained model of method 300 will compare the biopsy data 302 with the baseline data to determine the severity of the active inflammation 310. The inflammation may be minimal 312, mild 314, moderate 316, or severe 318. The severity of the inflammation may provide insight into the diagnosis and analysis of the accurate of IBD.

Moving now to FIG. 4. FIG. 4 provides an example method that may be performed using the computing device of FIG. 6, and is suitable for achieving the described advantages based on detecting disease activity associated with IBD, dysplasia, and adenocarcinoma in one or more patients. In embodiments, one or more computer storage media having computer-executable instructions embodied thereon that, when executed, by one or more processors, cause the one or more processors to perform operations illustrated in FIG. 4. Each block or step of method 300 and other methods described herein comprises a computing process that may be performed using any combination of hardware, firmware, and/or software. For instance, various functions may be carried out by a processor executing instructions stored in memory. The methods may also be embodied as computer-usable instructions stored on computer storage media. The methods may be provided by a stand-alone application, a service or hosted service (stand-alone or in combination with another hosted service), or a plug-in to another product, to name a few. Accordingly, method 400 may be performed by one or more computing devices, such as a smartphone or other user device, a server, or by a distributed computing platform, such as in the cloud. The method 400 of FIG. 4 may use the trained model described with respect to FIG. 3 or additional components, models, or methods described with respect to FIG. 4

The present technology disclosed herein also includes development of a BDNN to enable early detection of IBD associated dysplasia and adenocarcinoma. A Bayesian deep learning model is developed using ResNet, DenseNet, and EfficientNet architectures. These models make decisions (e.g., no dysplasia, low-grade/high-grade dysplasia) that are critical to better assess dysplasia and subsequent development of adenocarcinoma. The model accuracy can be evaluated using the labeled data and other clinical data associated with pathologist observations. From the labeled dataset, 70% can be used for model training, 15% for model validation, and 15% for model testing. Data augmentation can also be used. Accuracy of 90% or higher for the training data set is achievable using this method.

Any cases that may have been predicated incorrectly may be investigated for architectural changes to the evaluated deep networks to achieve even higher accuracy using the trained deep networks. Any image incorrectly processed by the trained model can be used for generating additional data via data augmentation for further training of the model.

The accurate histologic grading of IBD may be used to improve the interobserver agreement. For example, the determined histologic grading of IBD can lead to the achievement of higher rates of histologic remission with lower rates of clinical relapse, and substantial improvement in the quality of life for each patient in which the histologic grading has been performed. Further, aspects of the disclosed technology can also lead to optimization of maintenance therapy, decrease the rate of corticosteroid use, and decrease hospitalization. Early detection of IBD associated dysplasia will lead to decreased rates of adenocarcinoma, and thus, will save lives. As a result, the nation's healthcare cost can be reduced by millions of dollars annually.

Further, this approach leads to more precise clinical management of patients at an individual patient level as the AI-assisted histologic grading of the disease activity will be more objective with less inter-observer variability. Thus, this technology is aimed to advance healthcare delivery for patients and beyond.

For example, method 400 may receive biopsy data 302 and have already determined that the data presents as abnormal with a particular severity of inflammation. The trained model of method 400 or method 300 will then compare the biopsy date with baseline data to determine if the biopsy data presents dysplasia 402 indications. Dysplasia may present in a biopsy as abnormal cells. In one example, the biopsy data 302 may not be clear as to if there is dysplasia present and may indicate indefinite for dysplasia 412. In another example, the trained model of method 300 or method 400 may indicate the absence of dysplasia 406. In yet another example, the trained model may indicate that dysplasia is present 404.

Once dysplasia is determined to be present, the trained model will determine the degree of dysplasia based on a comparison of the biopsy data 302 with historical baseline data. An indication of low grade dysplasia 408 may present as an indication that adenocarcinoma is not likely in the patient. Additionally, the trained model may determine that the biopsy data 302 presents as high grade dysplasia 410. An indication that high grade dysplasia 410 is present may indicate that the biopsy data 302 suggests that there is a presence of adenocarcinoma. The method 300 or 400 may provide the results of the decision making tree within the user interface.

FIG. 5 provides an example method that may be performed using the computing device of FIG. 6, and is suitable for achieving the described advantages based on detecting disease activity associated with IBD, dysplasia, and adenocarcinoma in one or more patients. In embodiments, one or more computer storage media having computer-executable instructions embodied thereon that, when executed, by one or more processors, cause the one or more processors to perform operations illustrated in FIG. 5. Each block or step of method 300 and other methods described herein comprises a computing process that may be performed using any combination of hardware, firmware, and/or software. For instance, various functions may be carried out by a processor executing instructions stored in memory. The methods may also be embodied as computer-usable instructions stored on computer storage media. The methods may be provided by a stand-alone application, a service or hosted service (stand-alone or in combination with another hosted service), or a plug-in to another product, to name a few. Accordingly, method 500 may be performed by one or more computing devices, such as a smartphone or other user device, a server, or by a distributed computing platform, such as in the cloud.

In one example, at step 502, method 500 accesses a training data set of histopathology images from one or more patients having IBD. Further, step 504 provides for training a deep learning model with the training data set to generate a trained model, the trained model configured to classify a histopathology image. The trained model described herein may be a BDNN. The BDNN is able to model uncertainty in the predictions. It is created by inserting a Dense layer followed by a Dropout layer (used during training) into an existing deep learning model such as ResNet, DenseNet, and EfficientNet. The transformations may be homogeneous and a multi-branch architecture.

At step 506, method 500 processes, via the trained model, the histopathology image of a target patient. Further, the operations may determine the target patient is at risk for dysplasia based on processing the histopathology image of the target patient and the histologic assessment determination. Moreover, the processing of the histopathology image comprises performing patch-wise classification to identify patterns using a BDNN with Monte Carlo dropout. At step 508, method 500 provides, based on processing the histopathology image of the target patient, a histologic assessment determination associated with an IBD severity for display via a user interface.

Having described an overview of embodiments of the present technology, an example operating environment in which embodiments of the present technology may be implemented is described in order to provide a general context for various aspects of the present technology. Referring now to FIG. 6, in particular, an example operating environment for implementing embodiments of the present technology is shown and designated generally as computing device 600. Computing device 600 is but one example of a suitable computing environment and is not intended to suggest any limitation as to the scope of use or functionality of the technology. Neither should computing device 600 be interpreted as having any dependency or requirement relating to any one or combination of components illustrated.

The technology of the present disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc. refer to code that perform particular tasks or implement particular abstract data types. The technology may be practiced in a variety of system configurations, including hand-held devices, consumer electronics, general-purpose computers, more specialty computing devices, etc. The technology may also be practiced in distributed computing environments where tasks are performed by remote-processing devices that are linked through a communications network.

With reference to FIG. 6, computing device 600 includes bus 610 that directly or indirectly couples the following devices: memory 612, one or more processors 614, one or more presentation components 616, input/output ports 618, input/output components 620, and illustrative power supply 622. Bus 610 represents what may be one or more busses (such as an address bus, data bus, or combination thereof). Although the various blocks of FIG. 6 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. We recognize that such is the nature of the art, and reiterate that the diagram of FIG. 6 is merely illustrates an example computing device that can be used in connection with one or more embodiments of the present technology. Distinction is not made between such categories as “workstation,” “server,” “laptop,” “hand-held device,” etc., as all are contemplated within the scope of FIG. 6 and reference to “computing device.”

Computing device 600 typically includes a variety of computer-readable media. Computer-readable media can be any available media that can be accessed by computing device 600 and includes both volatile and nonvolatile media, removable and non-removable media. By way of example, and not limitation, computer-readable media may comprise computer storage media and communication media.

Computer storage media include volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 600. Computer storage media excludes signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 612 includes computer storage media in the form of volatile or nonvolatile memory. The memory may be removable, non-removable, or a combination thereof. Example hardware devices include solid-state memory, hard drives, optical-disc drives, etc. Computing device 600 includes one or more processors that read data from various entities such as memory 612 or I/O components 620. Presentation component(s) 616 present data indications to a user or other device. Examples of presentation components include a display device, speaker, printing component, vibrating component, etc.

I/O ports 618 allow computing device 600 to be logically coupled to other devices including I/O components 620, some of which may be built in. Illustrative components include a microphone, joystick, game pad, satellite dish, scanner, printer, wireless device, etc.

Embodiments described above may be combined with one or more of the specifically described alternatives. In particular, an embodiment that is claimed may contain a reference, in the alternative, to more than one other embodiment. The embodiment that is claimed may specify a further limitation of the subject matter claimed.

The subject matter of the present technology is described with specificity herein to meet statutory requirements. However, the description itself is not intended to limit the scope of this disclosure. Rather, the inventors have contemplated that the claimed or disclosed subject matter might also be embodied in other ways, to include different steps or combinations of steps similar to the ones described in this document, in conjunction with other present or future technologies. Moreover, although the terms “step” or “block” might be used herein to connote different elements of methods employed, the terms should not be interpreted as implying any particular order among or between various steps herein disclosed unless and except when the order of individual steps is explicitly stated.

For purposes of this disclosure, the word “including” has the same broad meaning as the word “comprising,” and the word “accessing” comprises “receiving,” “referencing,” or “retrieving.” Further the word “communicating” has the same broad meaning as the word “receiving,” or “transmitting” facilitated by software or hardware-based buses, receivers, or transmitters” using communication media described herein. Also, the word “initiating” has the same broad meaning as the word “executing or “instructing” where the corresponding action can be performed to completion or interrupted based on an occurrence of another action.

In addition, words such as “a” and “an,” unless otherwise indicated to the contrary, include the plural as well as the singular. Thus, for example, the constraint of “a feature” is satisfied where one or more features are present. Also, the term “or” includes the conjunctive, the disjunctive, and both (a or b thus includes either a or b, as well as a and b).

For purposes of a detailed discussion above, embodiments of the present technology described with reference to a distributed computing environment; however the distributed computing environment depicted herein is merely an example. Components can be configured for performing novel aspects of embodiments, where the term “configured for” can refer to “programmed to” perform particular tasks or implement particular abstract data types using code.

From the foregoing, it will be seen that this technology is one well adapted to attain all the ends and objects described above, including other advantages that are obvious or inherent to the structure. It will be understood that certain features and subcombinations are of utility and may be employed without reference to other features and subcombinations. This is contemplated by and is within the scope of the claims. Since many possible embodiments of the described technology may be made without departing from the scope, it is to be understood that all matter described herein or illustrated the accompanying drawings is to be interpreted as illustrative and not in a limiting sense.

What is claimed is:

1. A system for histological grading predictions for inflammatory bowel disease (IBD), the system comprising:
at least one processor; and
one or more computer storage media storing computer executable instructions that when executed by the at least one processor, cause the at least one processor to perform operations comprising:
accessing a training data set of histopathology images from one or more patients having IBD;
training a deep learning model with the training data set to generate a trained model, the trained model configured to classify a histopathology image;
processing, via the trained model, the histopathology image of a target patient; and
providing, based on processing the histopathology image of the target patient, a histologic assessment determination associated with an IBD severity for display via a user interface.

2. The system of claim 1, wherein the trained model is a bayesian deep neural network (BDNN).

3. The system of claim 1, wherein the operations further comprise determining the target patient is at risk for dysplasia based on processing the histopathology image of the target patient and the histologic assessment determination.

4. The system of claim 2, wherein the BDNN relies on inserting neuron layers before the final fully-connected layer.

5. The system of claim 4, further comprising a dense layer followed by a dropout layer being inserted into the BDNN.

6. The system of claim 1, wherein the processing the histopathology image of the target patient comprises performing patch-wise classification to identify patterns.

7. The system of claim 1, wherein the training the deep learning model further comprises using a data augmentation technique on histopathology images.

8. A method for histological grading predictions for inflammatory bowel disease (IBD), the system comprising:
accessing a training data set of histopathology images from one or more patients having IBD;
training a deep learning model with the training data set to generate a trained model, the trained model configured to classify a histopathology image;
processing, via the trained model, the histopathology image of a target patient; and
providing, based on processing the histopathology image of the target patient, a histologic assessment determination associated with a dysplasia severity for display via a user interface.

9. The method of claim 8, wherein the trained model is a Bayesian deep neural network (BDNN).

10. The method of claim 8, wherein the operations further comprise determining the target patient is at risk for dysplasia based on processing the histopathology image of the target patient and the histologic assessment determination.

11. The method of claim 9, wherein the BDNN relies on inserting neuron layers before the final fully-connected layer.

12. The method of claim 11, further comprising a dense layer followed by a dropout layer being inserted into the BDNN.

13. The method of claim 8, wherein the processing the histopathology image of the target patient comprises performing patch-wise classification to identify patterns.

14. The method of claim 8, wherein the training the deep learning model further comprises using a data augmentation technique on histopathology images.

15. One or more non-transitory computer storage media having computer-executable instructions embodied thereon, that when executed by at least one processor, cause operations comprising:
accessing a training data set of histopathology images from one or more patients having inflammatory bowel disease (IBD);
training a deep learning model with the training data set to generate a trained model, the trained model configured to classify a histopathology image, wherein the training the deep learning model further comprises using a data augmentation technique;
processing, via the trained model, the histopathology image of a target patient; and
providing, based on processing the histopathology image of the target patient, a histologic assessment determination associated with an IBD severity for display via a user interface.

16. The media of claim 15, wherein the trained model is a Bayesian deep neural network (BDNN).

17. The media of claim 15, wherein the operations further comprise determining the target patient is at risk for dysplasia based on processing the histopathology image of the target patient and the histologic assessment determination.

18. The media of claim 16, wherein the BDNN relies on inserting neuron layers before the final fully-connected layer.

19. The media of claim 18, further comprising a dense layer followed by a dropout layer being inserted into the BDNN.

20. The media of claim 15, wherein the processing the histopathology image of the target patient comprises performing patch-wise classification to identify patterns.
